(19) United States
(12) Patent Application Publication
(10) Pub. No.: US 2026/0047880 A1
(43) Pub. Date: Feb. 19, 2026

(54) MEDICAL INSTRUMENT SHAFT ASSEMBLY WITH NESTED COMPONENTS

(71) Applicant: Cilag GmbH International, Zug (CH)

(72) Inventors: Tyler N. Brehm, Arcanum, OH (US); Jason R. Lesko, Cincinnati, OH (US)

(21) Appl. No.: 18/804,243

(22) Filed: Aug. 14, 2024

(51) Int. Cl. A61B 18/14

(52) U.S. Cl. CPC A61B 18/1445

(57) ABSTRACT

An apparatus includes a body, a shaft assembly, and an end effector. The shaft assembly includes an outer shaft and two elongate members. A first portion of the first elongate member is positioned within a recess of the second elongate member, while a second portion of the first elongate member is laterally offset relative to the second elongate member. A first portion of the second elongate member is positioned within a recess of the first elongate member, while a second portion of the second elongate member is laterally offset relative to the first elongate member. The end effector includes a first component coupled with the first elongate member and a second a second component coupled with the second elongate member, such that the first and second elongate members are operable to drive movement of the respective first and second components of the end effector.

### BACKGROUND

A variety of surgical instruments include a tissue cutting element and one or more elements that transmit radio frequency (RF) energy to tissue (e.g., to coagulate or seal the tissue). Examples of such electrosurgical instruments and related concepts are disclosed in U.S. Pat. No. 6,500,176 entitled “Electrosurgical Systems and Techniques for Sealing Tissue,” issued Dec. 31, 2002, the disclosure of which is incorporated by reference herein, in its entirety; U.S. Pat. No. 9,526,565, entitled “Electrosurgical Devices,” issued Dec. 27, 2016, the disclosure of which is incorporated by reference herein, in its entirety; U.S. Pat. No. 11,857,247, entitled “Jaw for Surgical Instrument End Effector,” issued Jan. 2, 2024, the disclosure of which is incorporated by reference herein, in its entirety; and U.S. Pub. No. 2022/0008120, entitled “Electrosurgical Instrument with Floating Jaw Component,” published Jan. 13, 2022, the disclosure of which is incorporated by reference herein in its entirety.

An electrosurgical instrument may be powered by an external generator. Examples of such generators are disclosed in U.S. Pat. No. 8,986,302, entitled “Surgical Generator for Ultrasonic and Electrosurgical Devices,” issued Mar. 24, 2015, the disclosure of which is incorporated by reference herein, in its entirety.

Some electrosurgical instruments may be supported and driven by a robotic surgical system. Examples of robotically controlled electrosurgical instruments are disclosed in U.S. Pat. No. 11,576,738, entitled “Systems and Instruments for Tissue Sealing,” issued Feb. 14, 2023, the disclosure of which is incorporated by reference herein in its entirety; and U.S. Pub. No. 2022/0338891, entitled “Systems for Setting Jaw Gap in Surgical Tool End Effectors,” published Oct. 27, 2022, the disclosure of which is incorporated by reference herein in its entirety.

In any surgical instrument, and particularly those used in minimally invasive surgical procedures and/or robotic surgical procedures, there may be a need to provide several different components within a relatively small diameter. This may present challenges with respect to assembling the instrument in a manner that is time efficient and cost efficient. The inclusion of numerous shaft assembly components within a relatively small diameter may also present challenges with respect to mitigation of manufacturing tolerances and maximizing mechanical stability. For instance, some surgical instrument shaft assemblies may have one or more components that may tend to buckle during operation of the instrument, such that it may be desirable to include one or more features that prevent or otherwise mitigate such buckling. In the case of electrosurgical instruments, a shaft assembly may also need to safely accommodate one or more wires, flex circuits, and/or other electrically conductive components.

While a variety of surgical instruments have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements.

FIG. 1 depicts a schematic view of an example of an electrosurgical instrument

FIG. 2A depicts a perspective view of an end effector and distal portion of a shaft assembly of the instrument of FIG. 1, with the end effector in a closed configuration.

FIG. 2B depicts a perspective view of the end effector and distal portion of the shaft assembly of FIG. 2A, with the end effector in an open configuration and a knife in a proximal position.

FIG. 2C depicts a perspective view of the end effector and distal portion of the shaft assembly of FIG. 2A, with the end effector in the open configuration and the knife in a distal position.

FIG. 3A depicts a side elevation view of the end effector and distal portion of the shaft assembly of FIG. 2A, with an outer shaft of the shaft assembly omitted, and with the end effector in the closed configuration.

FIG. 3B depicts a side elevation view of the end effector and distal portion of the shaft assembly of FIG. 2A, with the outer shaft of the shaft assembly omitted, and with the end effector in the open configuration.

FIG. 4 depicts a perspective view of a first jaw of the end effector of the instrument of FIG. 1.

FIG. 5 depicts a perspective view of a second jaw of the end effector of the instrument of FIG. 1.

FIG. 6 depicts an exploded perspective view of the shaft assembly of the instrument of FIG. 1.

FIG. 7 depicts an exploded perspective view of a distal portion of the shaft assembly of the instrument of FIG. 1.

FIG. 8 depicts a perspective view of the distal portion of the shaft assembly of the instrument of FIG. 1.

FIG. 9 depicts an end view, oriented from the distal end toward the proximal end, of the shaft assembly of the instrument of FIG. 1.

FIG. 10 depicts a perspective view of the distal portion of an inner guide of the shaft assembly of the instrument of FIG. 1.

FIG. 11 depicts another perspective view of the inner guide of FIG. 10.

FIG. 12 depicts an end view, oriented from the distal end toward the proximal end, of the inner guide of FIG. 10.

FIG. 13 depicts a perspective view of the distal portion of a closure beam of the shaft assembly of the instrument of FIG. 1.

FIG. 14 depicts an end view, oriented from the distal end toward the proximal end, of the closure beam of FIG. 13.

FIG. 15 depicts an end view, oriented from the distal end toward the proximal end, of a knife assembly of the shaft assembly of the instrument of FIG. 1.

FIG. 16 depicts a perspective view of the knife assembly of FIG. 15.

FIG. 17A depicts an end view, oriented from the distal end toward the proximal end, of the shaft assembly of the instrument of FIG. 1 at a first stage of an assembly process.

FIG. 17B depicts an end view, oriented from the distal end toward the proximal end, of the shaft assembly of the instrument of FIG. 1 at a second stage of an assembly process.

FIG. 17C depicts an end view, oriented from the distal end toward the proximal end, of the shaft assembly of the instrument of FIG. 1 at a third stage of an assembly process.

FIG. 17D depicts an end view, oriented from the distal end toward the proximal end, of the shaft assembly of the instrument of FIG. 1 at a fourth stage of an assembly process.

FIG. 17E depicts an end view, oriented from the distal end toward the proximal end, of the shaft assembly of the instrument of FIG. 1 at a fifth stage of an assembly process.

FIG. 18 depicts an exploded perspective view of an example of an alternative knife assembly that may be incorporated into the shaft assembly of the instrument of FIG. 1.

FIG. 19 depicts a perspective view of a distal portion of the knife assembly of FIG. 18.

FIG. 20 depicts an end view, oriented from the proximal end toward the distal end, of the knife assembly of FIG. 18.

### DETAILED DESCRIPTION

Any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

### I. Overview of Electrosurgical Instrument

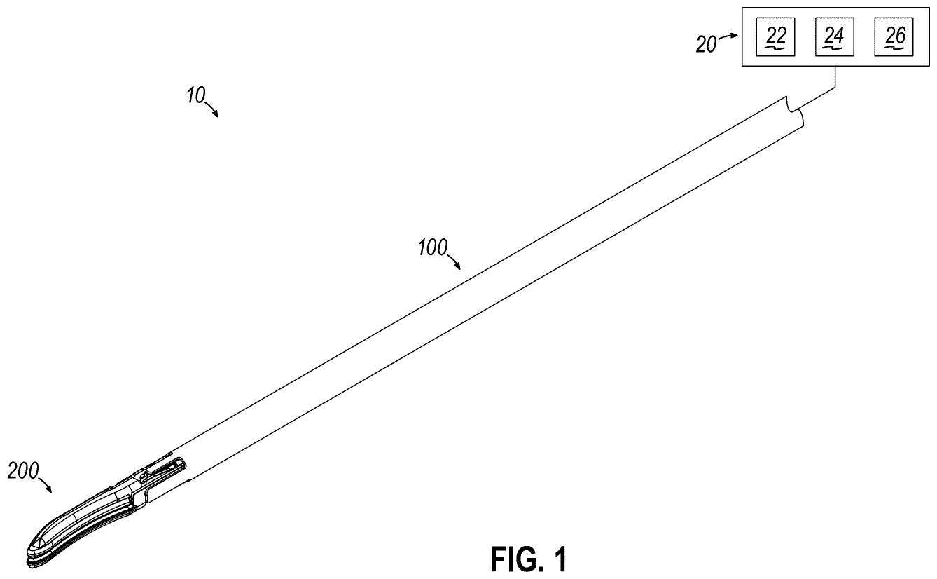

FIG. 1 shows an example of an electrosurgical instrument (10) that may be used in numerous kinds of medical procedures. Instrument (10) of this example includes a body assembly (20), a shaft assembly (100), and an end effector (200). Shaft assembly (100) extends distally relative to body assembly (20). End effector (200) is positioned at a distal end of shaft assembly (100). As will be described in greater detail below, end effector (200) is operable to grasp, cut, and seal or weld tissue (e.g., a blood vessel, etc.). Specifically, end effector (200) is operable to grasp tissue between opposing jaws (220, 240), cut the tissue with a knife (184), and seal or weld the tissue by applying bipolar RF energy to the tissue via respective electrodes (224, 244) of jaws (220, 240). While not shown in FIG. 1, instrument (10) may be coupled with a generator that is operable to provide the RF energy to electrodes (224, 244).

Body assembly (20) may take numerous different forms. In some versions, body assembly (20) includes a handle, such that body assembly (20) is configured to be grasped and manipulated by at least one hand of a human operator. By way of example only, such a handle may include a pistol grip, a scissor grip, or any other suitable configuration. In some other versions, body assembly (20) is configured to be coupled with a robotic arm or other component of a robotic surgical system, such that body assembly (20) is configured to be supported and activated by the robotic surgical system. Alternatively, body assembly (20) may take any other suitable form.

Body assembly (20) of the present example includes a jaw closure assembly (22), a knife drive assembly (24), and an input interface (26). Jaw closure assembly (22) is operable to drive jaws (220, 240) of end effector between an open position and a closed position, with closure motion from jaw closure assembly (22) being communicated to end effector (200) via shaft assembly (100) as will be described in greater detail below. Any suitable components and arrangements may be used to form jaw closure assembly (22) as will be apparent to those skilled in the art in view of the teachings herein. Knife drive assembly (24) is operable to drive knife (184) between a proximal position and a distal position, with translational motion from knife drive assembly (24) being communicated to end effector (200) via shaft assembly (100) as will be described in greater detail below. Any suitable components and arrangements may be used to form knife drive assembly (24) as will be apparent to those skilled in the art in view of the teachings herein.

Input interface (26) is operable to interface with a human or robotic operator to activate jaw closure assembly (22), knife drive assembly (24), and/or communication of RF energy to electrodes (224, 244). In versions where body assembly (30) is configured to be grasped and manipulated by at least one hand of a human operator, input interface (26) may include one or more trigger, one or more levers, one or more sliders, one or more buttons, and/or any other suitable kind(s) of input features, including combinations thereof. In versions where body assembly (30) is configured to be coupled with a robotic arm or other component of a robotic surgical system, input interface (26) may include one or more gears, one or more drive spindles, one or more shuttles or other translating drive elements, and/or other kind(s) of features that mechanically interface with complementary features of the robotic arm or other component of the robotic surgical system. In addition, or in the alternative, in versions where body assembly (30) is configured to be coupled with a robotic arm or other component of a robotic surgical system, input interface (26) may include one or more pins or sockets, one or more other kinds of electrical contacts, and/or any other kind(s) of features that electrically interface with complementary features of the robotic arm or other component of the robotic surgical system.

### A. Overview of End Effector

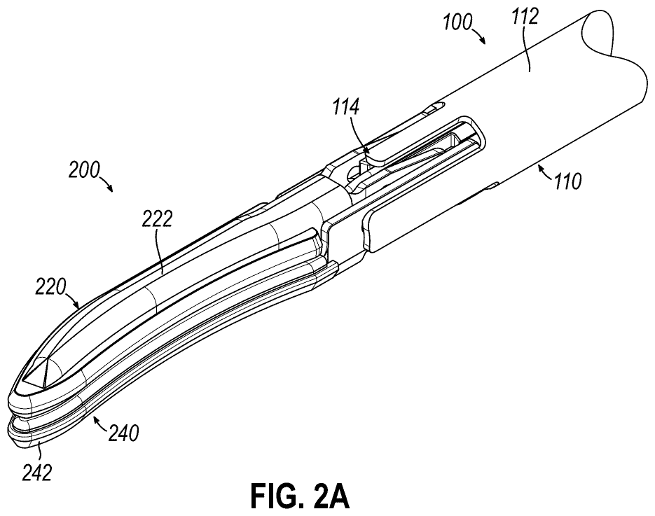

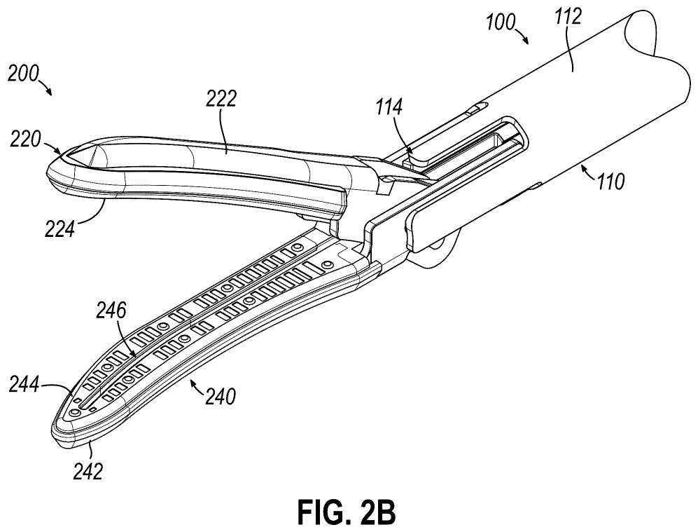

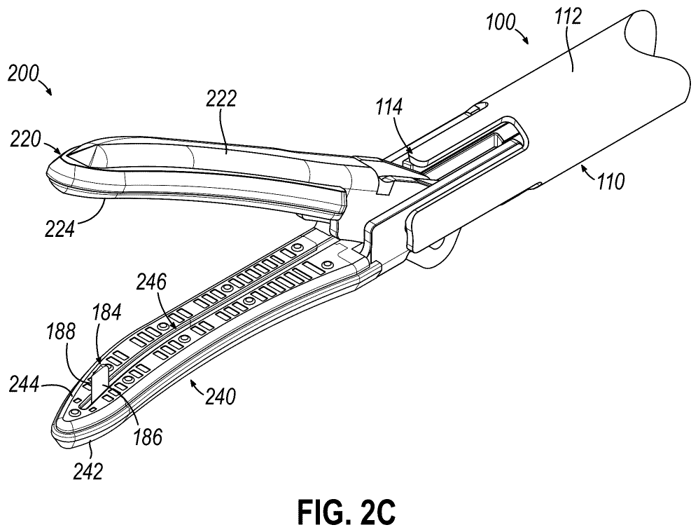

As shown in FIGS. 2A-2C, and as noted above, end effector (200) of the present example includes a first jaw (220) and a second jaw (240) that are operable to transition between a closed configuration (FIG. 2A) and an open configuration (FIG. 2B). In the present example, jaw (220) pivots toward and away from jaw (240), while jaw (240) remains stationary relative to shaft assembly (100), as jaws (220, 240) transition between the open configuration and the closed configuration. In some other versions, both jaws (220, 240) pivot simultaneously toward and away from each other in opposing fashion to transition between the open configuration and the closed configuration. Alternatively, one or both of jaws (220, 240) may move in any other suitable fashion.

As shown in the transition from FIG. 2B to FIG. 2C, knife (184) is operable to translate relative to jaws (220, 240) from a proximal position (FIG. 2B) to a distal position (FIG. 2C). While knife (184) is shown as being in the distal position while jaws (220, 240) are in the open configuration, some versions of operation may provide translation of knife (184) from the proximal position to the distal position only when jaws (220, 240) are in the closed configuration. In other words, in some versions of instrument (10) and methods of use of instrument (10), knife (184) would not be in the distal position when jaws (220, 240) are in the open configuration. The arrangement shown in FIG. 2C should therefore be understood as only being provided for purposes of illustration to show a distal position of knife (184) in relation to jaw (240).

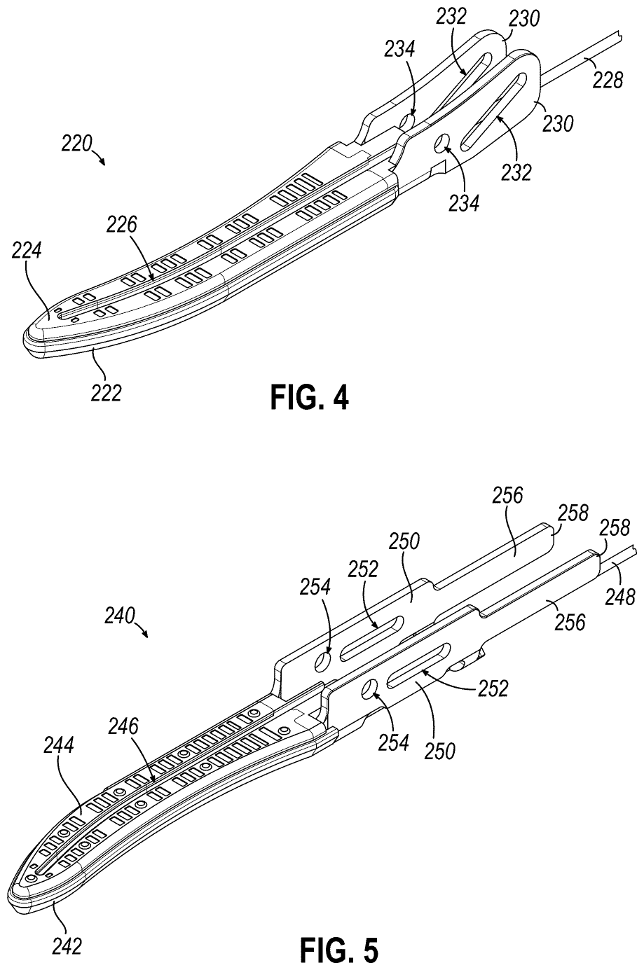

As shown in FIGS. 2A-4 jaw (220) includes a body (222) and an electrode (224). In versions where body (222) includes an electrically conductive material (e.g., metal, etc.), an electrically insulative material (e.g., polymer, etc.) may be interposed between body (222) and electrode (224) to electrically isolate the electrically conductive portion of body (222) relative to electrode (224). For instance, an insulative plastic material may be overmolded over a metallic portion of body (222) (or otherwise be secured to the metallic portion of body (222)), and electrode (224) may be secured to the other side of the plastic material, such that the plastic material is interposed between electrode (224) and the metallic portion of body (222). In the present example, body (222) and electrode (224) curve laterally away from a central longitudinal axis along a distal portion of the length of jaw (220). In some other versions, these features are substantially straight along the entire length of jaw (220).

Jaw (220) defines a knife slot (226) passing through electrode (224). Knife slot (226) is configured to accommodate longitudinal motion of knife (184) relative to jaw (220) while jaws (220, 240) are in the closed configuration. Jaw (220) further includes a pair of proximal flanges (230) which are laterally spaced apart from each other. Each flange (230) defines an elongate slot (232) and a pin opening (234). Each elongate slot (232) is oriented obliquely along its corresponding flange (230), which provides closure motion to jaw (220) as described in greater detail below. A wire (228) extends proximally from jaw (220). Wire (228) is coupled with electrode (224) and extends along the length of shaft assembly (100) as described in greater detail below. Wire (228) is further configured to couple with a source of RF energy (e.g., an external generator), such that wire (228) is configured to provide RF energy to electrode (224).

As shown in FIGS. 2A-3 and 5 jaw (240) includes a body (242) and an electrode (244). In versions where body (242) includes an electrically conductive material (e.g., metal, etc.), an electrically insulative material (e.g., polymer, etc.) may be interposed between body (242) and electrode (244) to electrically isolate the electrically conductive portion of body (242) relative to electrode (244). For instance, an insulative plastic material may be overmolded over a metallic portion of body (242) (or otherwise be secured to the metallic portion of body (242)), and electrode (244) may be secured to the other side of the plastic material, such that the plastic material is interposed between electrode (244) and the metallic portion of body (242). In the present example, body (242) and electrode (244) curve laterally away from a central longitudinal axis along a distal portion of the length of jaw (240). In some other versions, these features are substantially straight along the entire length of jaw (240).

Jaw (240) defines a knife slot (246) passing through electrode (244). Knife slot (246) is configured to accommodate longitudinal motion of knife (144) relative to jaw (240). Jaw (240) further includes a pair of proximal flanges (250) which are laterally spaced apart from each other. Each flange (250) defines an elongate slot (252) and a pin opening (254). Each elongate slot (252) is oriented longitudinally along its corresponding flange (250). A beam (256) extends proximally from each flange (250) and terminates in a respective proximal face (258). A wire (248) extends proximally from jaw (240). Wire (248) is coupled with electrode (244) and extends along the length of shaft assembly (100) as described in greater detail below. Wire (248) is further configured to couple with a source of RF energy (e.g., an external generator), such that wire (248) is configured to provide RF energy to electrode (244).

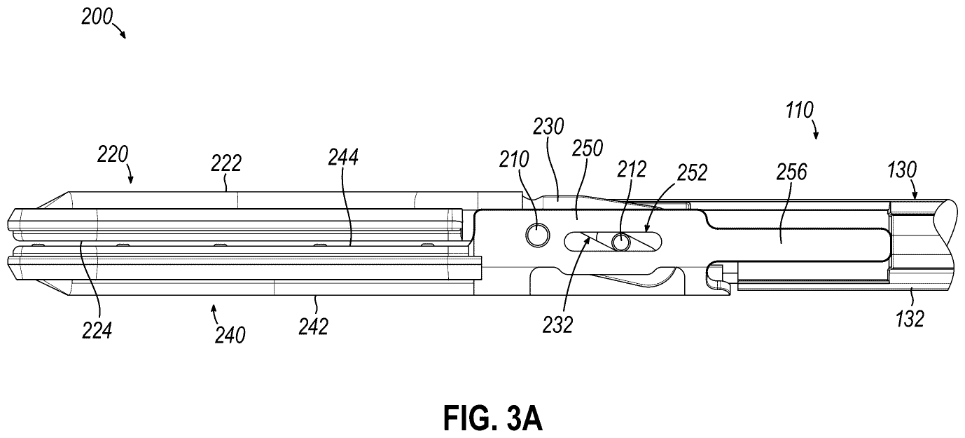

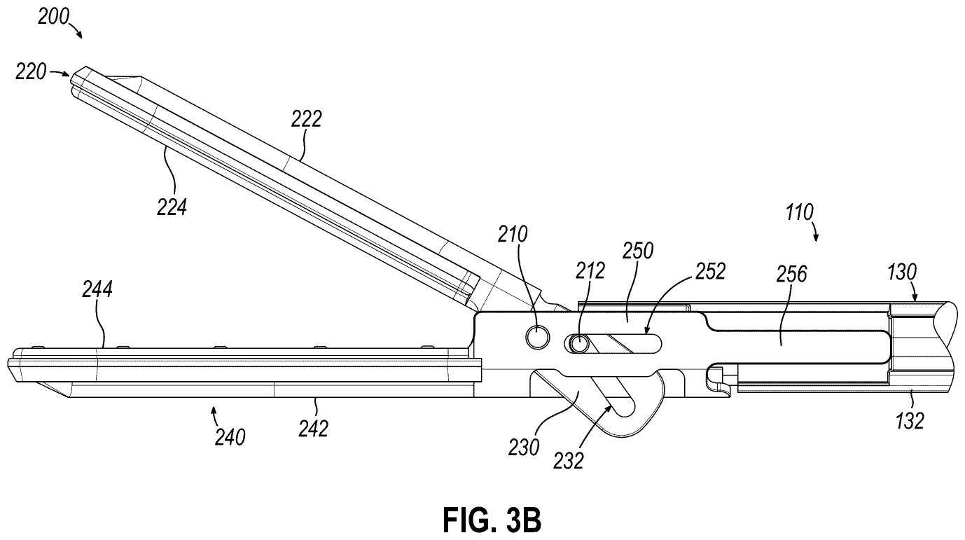

As shown in FIGS. 3A-3B, a pin (210) is disposed through openings (234, 254) of flanges (230, 250) of jaws (220, 240), thereby pivotably coupling jaws (220, 240) to each other. Another pin (212) is disposed through slots (232, 252). Pin (212) is operable to be driven between a proximal position (FIG. 3A) and a distal position (FIG. 3B) as will be described in greater detail below. Because slots (232) of flanges (230) are oriented obliquely while slots (252) of flanges (250) are oriented longitudinally in this example, longitudinal motion of pin (212) along slots (232, 252) will drive pivotal motion of jaws (220, 240). In particular, pin (212) will drive jaw (220) to a closed position when pin (212) is in a proximal position (FIG. 3A); and to an open position when pin (212) is in a distal position (FIG. 3B). Alternatively, jaws (220, 240) may be driven between open and closed configurations using any other suitable features or techniques.

In use, tissue (e.g., a vessel, etc.) may be positioned between jaws (220, 240) while jaws (220, 240) are in an open configuration. Then, jaws (220, 240) may be driven to the closed configuration to clamp the tissue. While jaws (220, 240) remain closed, knife (184) may be driven from the proximal position to the distal position to sever the tissue. In addition, bipolar RF energy may be applied to the tissue via electrodes (224, 244) to thereby seal the tissue. In some versions, electrode (244) serves as an active electrode while electrode (224) serves as a return electrode. Alternatively, this relationship may be reversed. In some examples of use, bipolar RF energy may be applied to the tissue via electrodes (224, 244) without knife (184) being advanced distally through the tissue. In either scenario, jaws (220, 240) may be returned to the open configuration to release the tissue when appropriate.

### B. Overview of Shaft Assembly

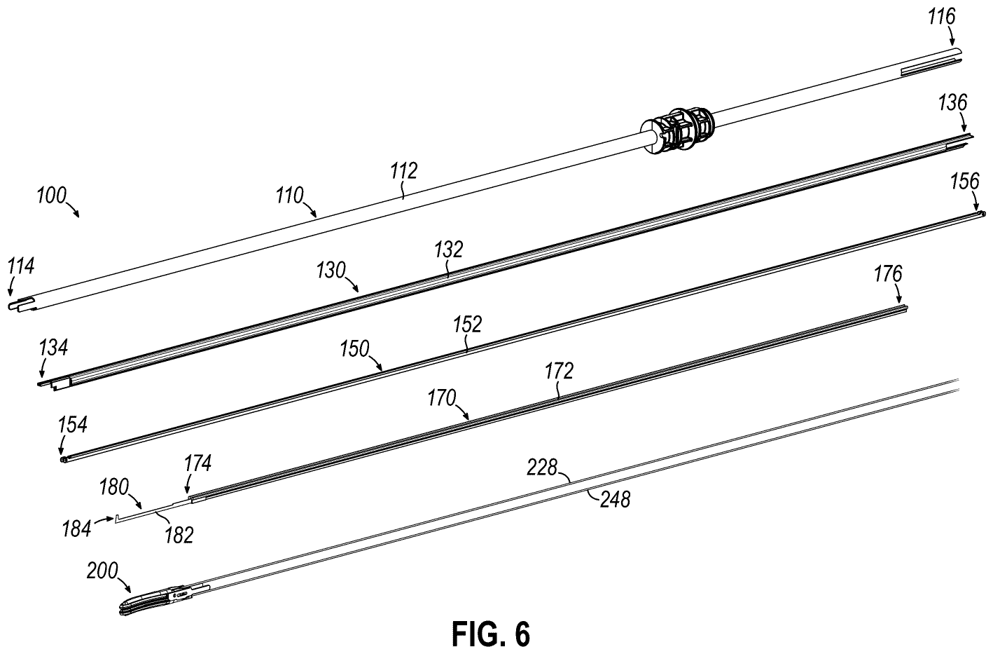

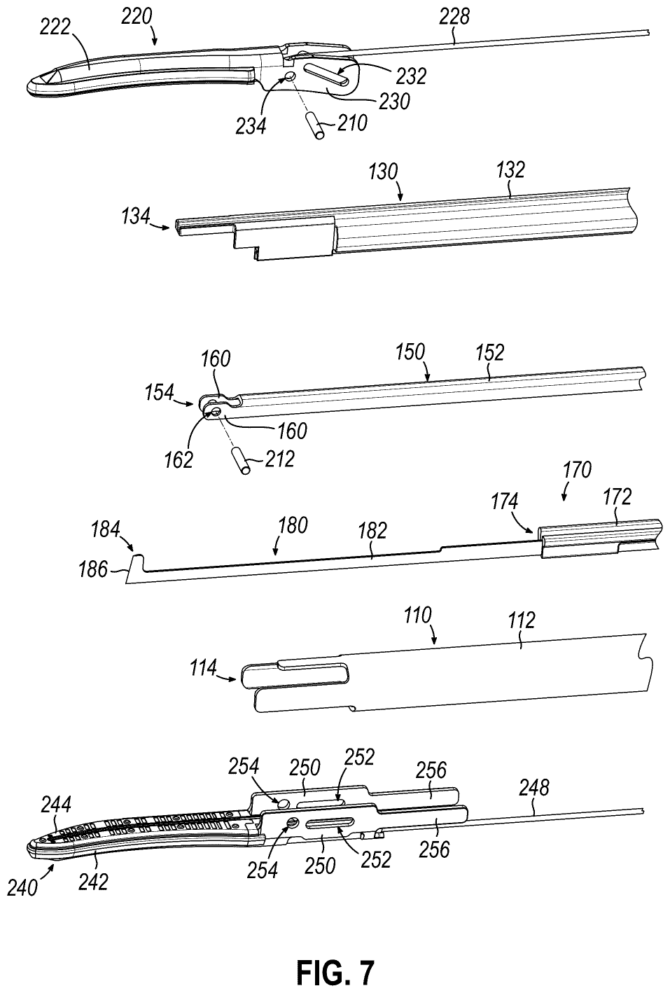

FIGS. 6-7 show components of shaft assembly (100) in greater detail. As shown, shaft assembly (100) of this example includes an outer shaft (110), an inner guide (130), a closure beam (150), and a knife assembly (170). Outer shaft (110) of this example includes a hollow tubular body (112) with a distal end (114) and a proximal end (116). End effector (200) is positioned at distal end (114) while proximal end (116) is disposed in body (20). Inner guide (130) of this example includes an elongate body (132) with a distal end (134) and a proximal end (136). Elongate body (132) of inner guide (130) is disposed within tubular body (112) of outer shaft (110). Elongate body (132) is laterally offset relative to the central longitudinal axis of tubular body (112) (i.e., not centered along the central longitudinal axis of tubular body (112)). Elongate body (132) of inner guide (130) remains stationary relative to tubular body (112) of outer shaft (110) during operation of instrument (10) in the present example. End effector (200) is positioned at distal end (134) while proximal end (136) is disposed in body (20). Wires (228, 248) are interposed between elongate body (132) of inner guide (130) and the inner surface of tubular body (112) of outer shaft (110) in this example.

Closure beam (150) of this example includes an elongate body (152) with a distal end (154) and a proximal end (156). Elongate body (152) of closure beam (150) is disposed within tubular body (112) of outer shaft (110), laterally adjacent to elongate body (132) of inner guide (130). In the present example, elongate body (152) is radially centered along the central longitudinal axis of tubular body (112). Elongate body (152) of the present example has an upside-down “U” shaped cross-sectional profile, though elongate body (152) may alternatively have any other suitable cross-sectional profile. Proximal end (156) is disposed within body (20) and is coupled with jaw closure assembly (22), such that jaw closure assembly (22) is operable to drive longitudinal translation of closure beam (150) relative to outer shaft (110) and relative to inner guide (130). Distal end (154) includes a clevis arrangement formed by two flanges (160), each flange (160) having a respective pin opening (162). Pin (212) is disposed in openings (162), in addition to being disposed in slots (232, 252) of flanges (230, 250) of jaws (220, 240). Thus, closure beam (150) drives translation of pin (212) to thereby drive opening and closing of jaws (220, 240) as described above. Alternatively, any other suitable components and arrangements may be used to drive opening and closing of jaws (220, 240).

Knife assembly (170) of the present example includes an elongate body (172) and a knife beam (180). Knife beam (180) is fixedly secured relative to elongate body (172) and extends distally relative to a distal end (174) of elongate body (172). Knife beam (180) includes a beam body (182) that distally terminates in knife (184). Knife (184) includes an upright portion (186) having a distal cutting edge (188). Elongate body (172) of knife assembly (170) is disposed within tubular body (112) of outer shaft (110), laterally adjacent to elongate body (132) of inner guide (130) and elongate body (152) of closure beam (150). Elongate body (172) is laterally offset relative to the central longitudinal axis of tubular body (112) (i.e., not centered along the central longitudinal axis of tubular body (112)), though the central longitudinal axis of tubular body (112) extends along a portion of elongate body (172) in this example. Elongate body (172) of the present example has a “U” shaped cross-sectional profile, though elongate body (152) may alternatively have any other suitable cross-sectional profile. In the present example, the “U” shape of elongate body (172) is partially nested within the “U” shape of elongate body (152).

A proximal end (176) of elongate body (172) is disposed within body (20) and is coupled with knife drive assembly (24), such that knife drive assembly (24) is operable to drive longitudinal translation of knife assembly (170) relative to outer shaft (110) and relative to inner guide (130). In the present example, knife assembly (170) and closure beam (150) are translatable independently relative to each other. Also in the present example, knife assembly (170) and knife drive assembly (24) are configured such that when knife (184) is at the distal-most position of the range of motion of knife (184) cutting edge (188) is spaced proximally from the distal ends of knife slots (226, 246). In other words, cutting edge (188) will not contact jaws (220, 240) at the distal ends of knife slots (226, 246) when knife (184) reaches the distal-most position of the range of motion of knife (184). This may prevent premature dulling of cutting edge (188) that might otherwise occur if cutting edge (188) were allowed to contact jaws (220, 240) at the distal ends of knife slots (226, 246) when knife (184) reaches the distal-most position of the range of motion of knife (184). In some other versions, knife assembly (170) and knife drive assembly (24) are configured to allow cutting edge (188) to contact jaws (220, 240) at the distal ends of knife slots (226, 246) when knife (184) reaches the distal-most position of the range of motion of knife (184).

### II. Example of Nesting Shaft Assembly Arrangement and Method of Assembly

As noted above, the inclusion of several components within a shaft assembly may present challenges with respect to assembling an instrument in a manner that is time efficient and cost efficient. The inclusion of numerous shaft assembly components within a relatively small diameter (e.g., approximately 5 mm to approximately 10 mm) may also present challenges with respect to mitigation of manufacturing tolerances and maximizing mechanical stability (e.g., avoiding buckling during operation of the instrument). In the case of an electrosurgical instrument such as instrument (10) described above, a shaft assembly may also need to safely accommodate one or more wires, flex circuits, and/or other electrically conductive components. The challenges associated with small diameter shaft assemblies may be exacerbated when the shaft assembly is substantially long (e.g., approximately 25 cm to approximately 45 cm). In the present example, shaft assembly (100) of instrument (10) may provide some or all these features and functionalities through the structural arrangement of the components of shaft assembly (100).

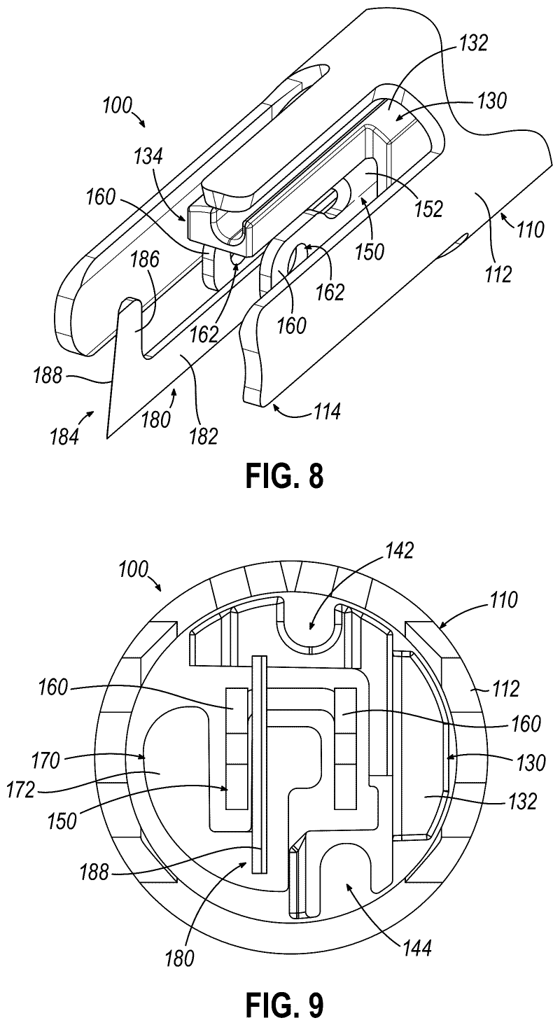

As best seen in FIGS. 8-9 and FIG. 17E, in which wires (228, 248) are omitted for clarity, the internal components (130, 150, 170) of shaft assembly (100) are contained within outer shaft (110) in a carefully nested arrangement, with minimal gaps provided between those internal components (130, 150, 170); and with minimal gaps provided between the inner surface (111) of tubular body (112) and exterior surfaces of the internal components (130, 150, 170). In addition to providing an efficient use of space within the interior of tubular body (112), the structural configurations of the internal components (130, 150, 170) also provide a self-supporting nesting arrangement providing robust structural integrity while also facilitating an efficient assembly process. The structural configurations and features of each internal component (130, 150, 170) that provide the above-noted functionalities will be described in greater detail below.

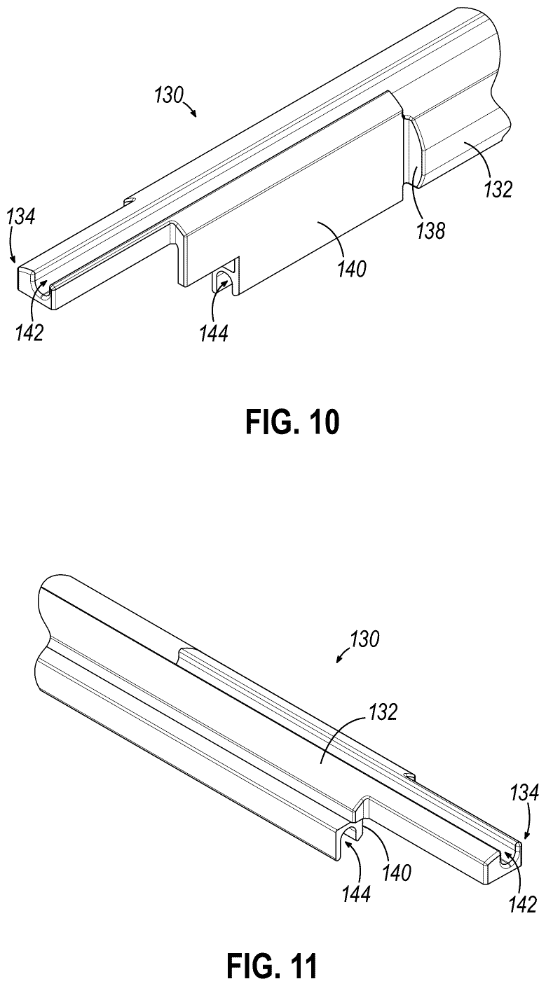

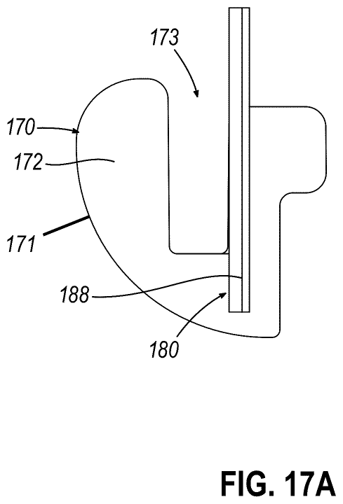

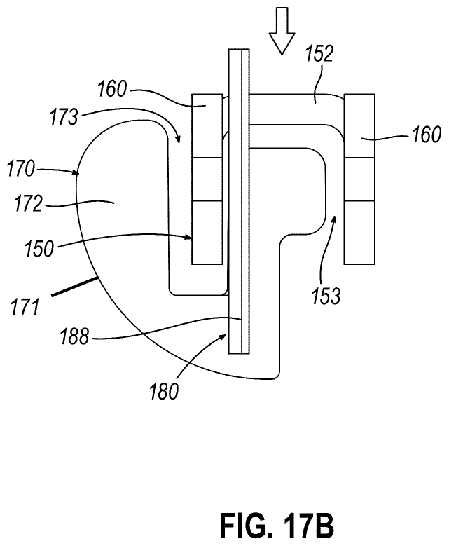

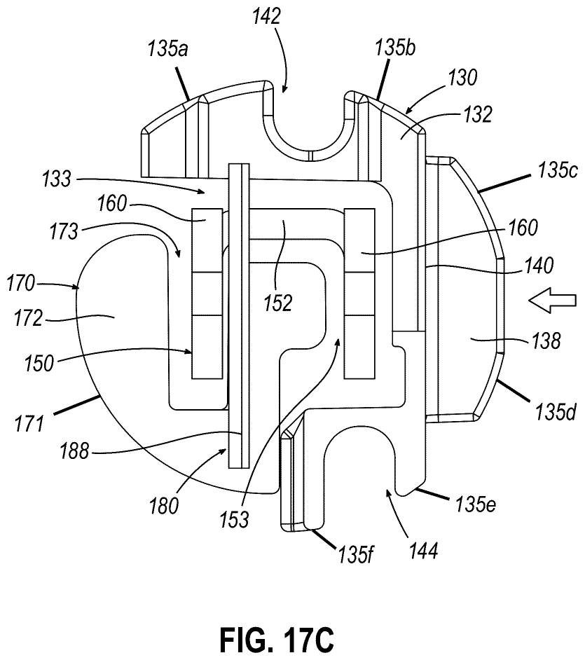

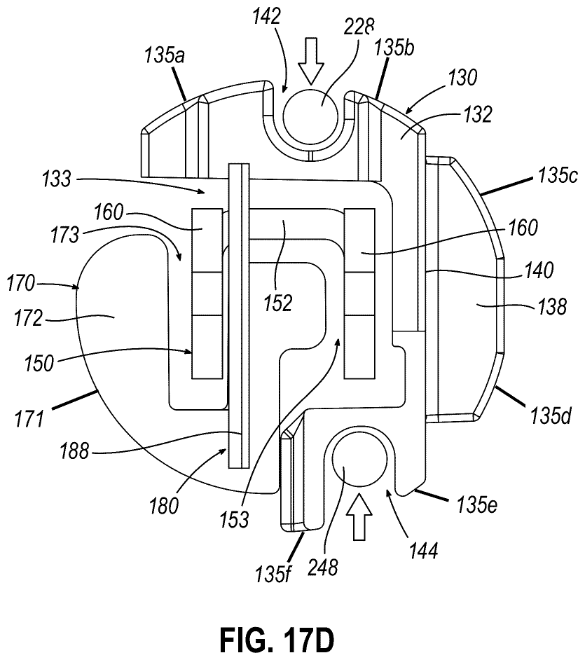

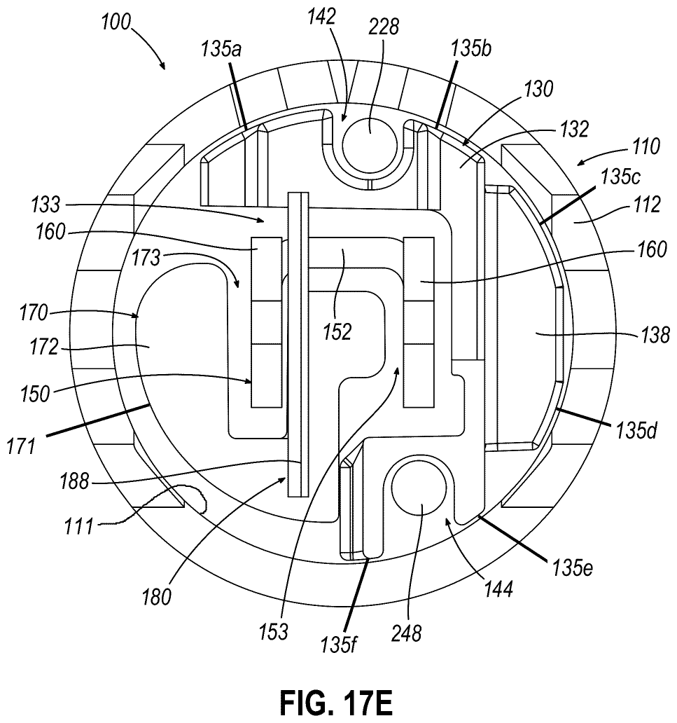

As shown in FIGS. 10-12, elongate body (132) of inner guide (130) includes a first channel (142) and a second channel (144), with each channel (142, 144) extending along the full length of elongate body (132). As best seen in FIGS. 17D-17E and as will be described in greater detail below, channel (142) is sized and positioned to receive wire (228) while channel (144) is sized and positioned to receive wire (248). As best seen in FIG. 10, elongate body (132) further includes a flat, laterally facing surface (140) near distal end (134), with a distally facing surface (138) positioned near the proximal end of laterally facing surface (140). These surfaces (138, 140) cooperate with the inner surface of tubular body (112) of outer shaft (110) to accommodate a beam (256) extending proximally from the corresponding flange (250) of jaw (240).

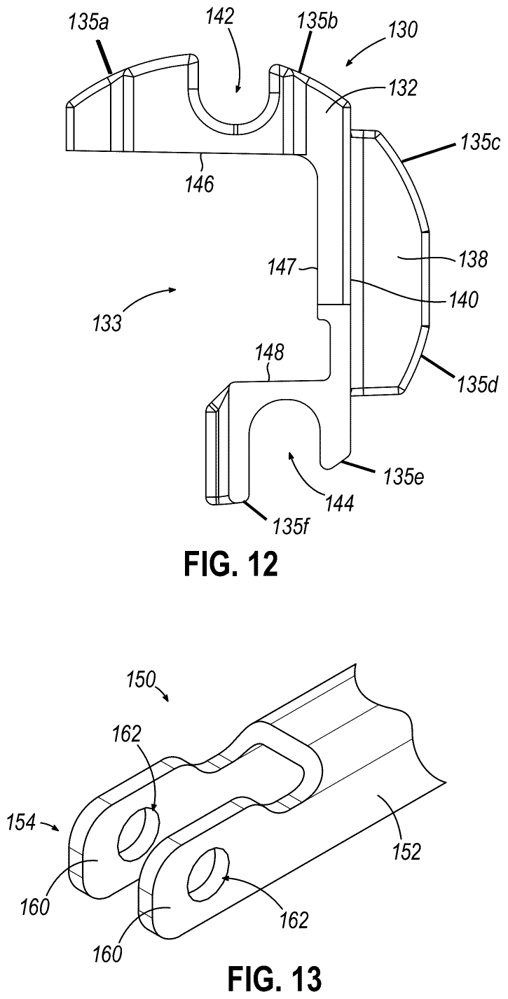

As best seen in FIG. 12, elongate body (132) has a cross-sectional profile that is generally “C” shaped or sideways-“U” shaped. This cross-sectional profile is provided in part by a set of internal surfaces (146, 147, 148) that cooperate to define an inwardly extending recess (133) or channel. In the present example, internal surfaces (146, 148) are parallel to each other and face each other, while internal surface (147) is perpendicular to internal surfaces (146, 148). Alternatively, internal surfaces (146, 147, 148) may have other orientations relative to each other. As described in greater detail below, recess (133) is sized and positioned to accommodate the combination of closure beam (150) and knife assembly (170). Elongate body (132) further includes curved outer surfaces (135) that are contoured to complement the radius of the inner surface (111) of tubular body (112). Outer surfaces (135a, 135b) are adjacent to first channel (142). Outer surfaces (135c, 135d) are adjacent to distally facing surface (138). Outer surfaces (135e, 135f) are adjacent to second channel (144).

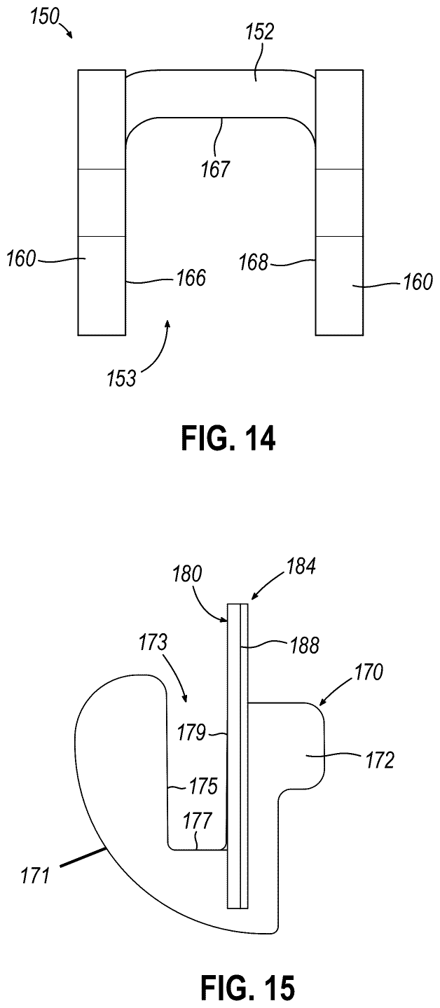

As noted above, and as shown in FIGS. 13-14, elongate body (152) of closure beam (150) has an upside-down “U” shaped cross-sectional profile. This cross-sectional profile is provided in part by a set of internal surfaces (166, 167, 168) that cooperate to define an inwardly extending recess (153) or channel. In the present example, internal surfaces (166, 168) are parallel to each other and face each other, while internal surface (167) is perpendicular to internal surfaces (166, 168). Alternatively, internal surfaces (166, 167, 168) may have other orientations relative to each other. As described in greater detail below, recess (153) is sized and positioned to accommodate a portion of knife assembly (170).

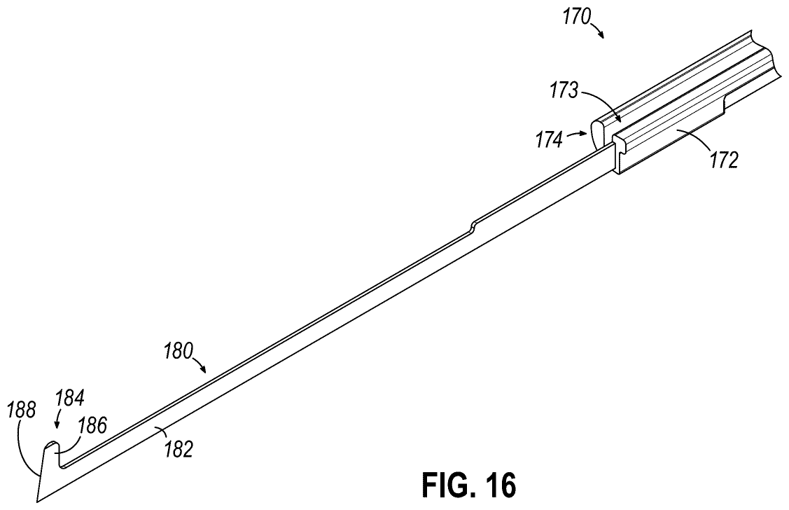

As noted above and as shown in FIGS. 15-16, elongate body (172) of knife assembly (170) has a “U” shaped cross-sectional profile. This cross-sectional profile is provided in part by a set of internal surfaces (175, 177, 179) that cooperate to define an inwardly extending recess (173) or channel. In the present example, two of the internal surfaces (175, 179) are parallel to each other and face each other, while internal surface (177) is perpendicular to the other internal surfaces (175, 179). Alternatively, internal surfaces (175, 177, 179) may have other orientations relative to each other. As described in greater detail below, recess (173) is sized and positioned to accommodate a portion of closure beam (150). Elongate body (172) further includes a curved outer surface (171) that is contoured to complement the radius of the inner surface (111) of tubular body (112).

FIGS. 17A-17E show examples of different stages of an assembly process of shaft assembly (100). In the present example, this process begins with knife assembly (170) by itself, as shown in FIG. 17A. Next, closure beam (150) is moved toward knife assembly (170) along a path of movement, as shown in FIG. 17B. In the present example, this path of movement is radially oriented or transversely oriented relative to the central longitudinal axis defined by the fully assembled shaft assembly (100). This path of movement of closure beam (150) is also transversely oriented relative to the central longitudinal axis defined by closure beam (150).

During this stage of assembly shown in FIG. 17B, a portion of elongate body (152) of closure beam (150) enters recess (173) of knife assembly (170); while a portion of elongate body (172) of knife assembly (170) enters recess (153) of closure beam (150). Knife assembly (170) and closure beam (150) thus fit together in an overlapping arrangement. While a portion of elongate body (152) of closure beam (150) is positioned in recess (173) of knife assembly (170), and a portion of elongate body (172) of knife assembly (170) is positioned in recess (153) of closure beam (150), there is still sufficient clearance between closure beam (150) and knife assembly (170) to allow closure beam (150) and knife assembly (170) to translate longitudinally independently of each other during operation of instrument (10) after the assembly of shaft assembly (100) is complete.

As shown in FIG. 17C, in a next stage of assembly, inner guide (130) is moved toward the combination of closure beam (150) and knife assembly (170) along a path of movement that is radially oriented or transversely oriented relative to the central longitudinal axis defined by the fully assembled shaft assembly (100). This path of movement of inner guide (130) is also transversely oriented relative to the central longitudinal axis defined by inner guide (130). In the present example, this path of inner guide (130) movement during the stage shown in FIG. 17C is also perpendicular to the path of closure beam (150) movement during the stage shown in FIG. 17B.

During the stage of assembly shown in FIG. 17C, a portion of the combination of closure beam (150) and knife assembly (170) enters recess (133) of inner guide (130). While portions of closure beam (150) and knife assembly (170) are positioned recess (173) of knife assembly (170), there is still sufficient clearance between closure beam (150), knife assembly (170), and inner guide (130) to allow closure beam (150) and knife assembly (170) to translate longitudinally relative to inner guide (130) during operation of instrument (10) after the assembly of shaft assembly (100) is complete.

Next, as shown in FIG. 17D, wire (228) is placed in channel (142) of inner guide (130) while wire (248) is placed in channel (144) of inner guide (130). Each wire (228, 248) may be placed in its corresponding channel (142, 144) along a respective insertion path transversely oriented relative to the central longitudinal axis defined by the respective wire (228, 248). This transversely oriented insertion path for each wire (228, 248) into its corresponding channel (142, 144) may substantially facilitate the manufacturing process, particularly in scenarios where wires (228, 248) are flexible. For instance, in other variations where wires (228, 248) are advanced longitudinally along corresponding channels (142, 144), it may be relatively difficult to push wires (228, 248) when wires (228, 248) lack substantial column strength, as such pushing of wires (228, 248) may result in a tendency for wires (228, 248) to buckle or bunch up within corresponding channels (142, 144) as wires (228, 248) are being pushed longitudinally along channels (142, 144). Moreover, in some cases wires (228, 248) may skive along an edge of outer shaft (110) and/or other components in scenarios where wires (228, 248) are advanced longitudinally along corresponding channels (142, 144). By contrast, a transversely oriented insertion path for each wire (228, 248) may allow for laying of the entire length of each wire (228, 248) along the length of the corresponding channel (142, 144), without the flexibility of wires (228, 248) creating any difficulties during this process, and without the risk of wires (228, 248) skiving along edges.

With all the above-noted internal components (130, 150, 170, 228, 248) of shaft assembly (100) arranged as shown in FIG. 17D, outer shaft (110) is positioned by sliding tubular body (112) over the combination of internal components (130, 150, 170, 228, 248). In the present example, this final positioning of tubular body (112) over the combination of internal components (130, 150, 170, 228, 248) is the only assembly motion that is provided along a longitudinal path that is (i.e., along the central longitudinal path axis of shaft assembly (100) or along a path that is parallel with the central longitudinal path axis of shaft assembly (100)). All the other paths of motion for internal components (130, 150, 170, 228, 248) during the assembly process are transversely oriented relative to the central longitudinal path axis of shaft assembly (100). This transverse motion during assembly may facilitate the assembly process, making it unnecessary to perform what may be more difficult steps of feeding relatively long components along longitudinal paths during the assembly process. It should nevertheless be understood that any of the insertion paths described above as being transversely oriented may in fact instead be longitudinally oriented in some variations of the process of manufacturing instrument (10).

As shown in FIGS. 8-9 and 17E, inner guide (130) is laterally offset from the central longitudinal axis of shaft assembly (100), knife assembly (170) is laterally offset from the central longitudinal axis of shaft assembly (100), and closure beam (150) is substantially radially centered relative to the central longitudinal axis of shaft assembly (100) in this example. As also shown in FIGS. 8-9 and 17E, inner guide (130), closure beam (150), and knife assembly (170) are closely nested with each other and relative to tubular body (112). This nesting arrangement may allow inner guide (130), closure beam (150), knife assembly (170), and outer shaft (110) to provide structural support to each other, to thereby reduce the risk of any of these components buckling during operation of instrument (10). For instance, the nesting arrangement between closure band (150) and knife assembly (170) may allow closure band (150) to prevent buckling of knife assembly (170) during operation of instrument (10); and may allow knife assembly (170) to prevent buckling of closure band (150) during operation of instrument (10). Such buckling of knife assembly (170) might otherwise tend to occur as knife assembly (170) is advanced distally; and such buckling of closure band (150) might otherwise tend to occur as closure band (150) is advanced distally.

Moreover, the containment of wires (228, 248) within respective channels (142, 144), adjacent to the inner surface of tubular body (112), substantially protects wires (228, 248) and isolates wires (228, 248) from movement of closure beam (150) and knife assembly (170) during operation of instrument (10). The complementary contours of outer surfaces (135, 171) and inner surface (111) minimize the gaps defined between tubular body (112), elongate body (132) of inner guide (130), and elongate body (172) of knife assembly (170). The minimization of such gaps may enhance the robustness of shaft assembly (100), minimize the degree of fluid infiltration within the interior of shaft assembly (100), and/or provide other effects. It should also be understood that the configuration and arrangement of internal components (130, 150, 170, 228, 248) in shaft assembly (100) may provide a lower part count than might otherwise be needed in other electrosurgical instrument shaft assembly configurations.

### III. Example of Alternative Knife Assembly

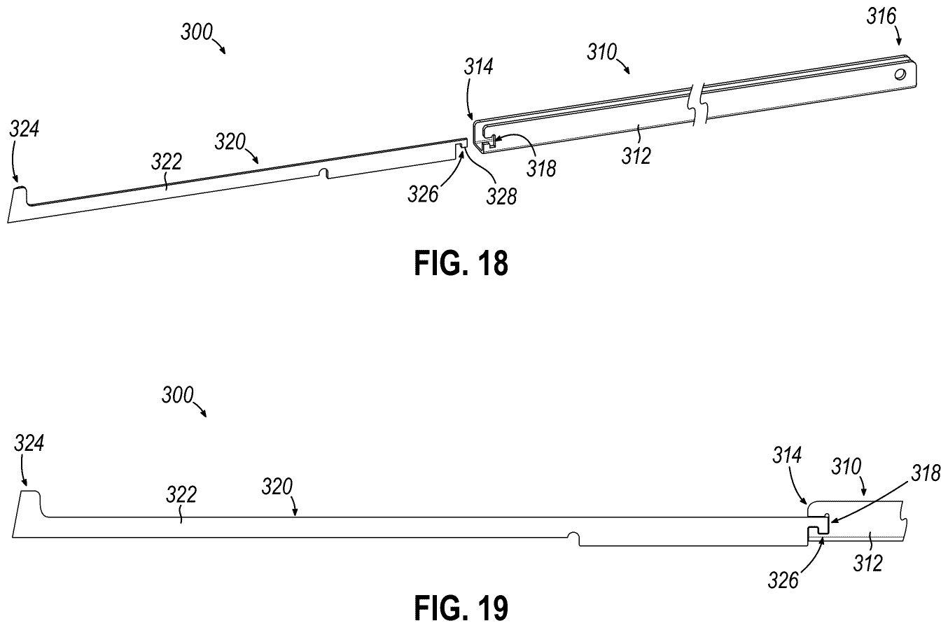

FIGS. 18-20 show an example of an alternative knife assembly (300) that may be incorporated into shaft assembly (100), in place of knife assembly (170). Knife assembly (300) of this example includes an elongate body (312) and a knife beam (320). Knife beam (320) is fixedly secured relative to elongate body (312) and extends distally relative to a distal end (314) of elongate body (312). Knife beam (320) includes a beam body (322) that distally terminates in a knife (324). Knife (324) may be configured and operable like knife (184) described above. A proximal end (326) of knife beam (320) includes a tab (328). Tab (328) is configured to fit in a complementary notch (318) formed at distal end (314) of elongate body (312). In some versions, tab (328) is inserted into notch (318), and then knife beam (320) is welded to elongate body (312). In some other versions, knife beam (320) and elongate body (312) are unitarily formed together as a monolithic homogenous continuum of material. Some such versions of knife assembly (300) may be formed using a stamping process and/or any other suitable process(es).

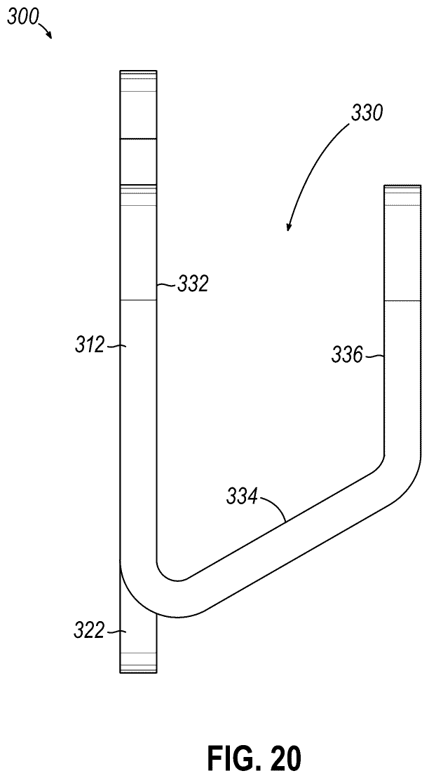

As best shown in FIG. 20, elongate body (312) the present example has a “U” shaped cross-sectional profile. This cross-sectional profile is provided by set of internal surfaces (332, 334, 336) that cooperate to define an inwardly extending recess (330). Like elongate body (172) of knife assembly (170), elongate body (312) of knife assembly (300) would be disposed within tubular body (112) of outer shaft (110), laterally adjacent to elongate body (132) of inner guide (130) and elongate body (152) of closure beam (150). Also like elongate body (172) of knife assembly (170), a portion of elongate body (312) of knife assembly (300) would be nested within recess (152) of closure beam (150). Similarly, a portion of closure beam (150) would be nested within recess (330) of elongate body (312). Knife assembly (300) may thus fit within shaft assembly (100) similar to the fit of knife assembly (170). Moreover, a proximal end (316) of elongate body (312) may be coupled with knife drive assembly (24), such that knife assembly (300) may be operated similar to knife assembly (170).

### IV. Examples of Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus comprising: (a) a body; (b) a shaft assembly extending distally relative to the body, the shaft assembly including: (i) an outer shaft, (ii) a first elongate member positioned within the outer shaft, the first elongate member being operable to translate relative to the outer shaft, the first elongate member defining a first recess, and (ii) a second elongate member positioned within the outer shaft, the second elongate member being operable to translate relative to the outer shaft and relative to the first elongate member, the second elongate member defining a second recess, a first portion of the first elongate member being positioned within the second recess, a second portion of the first elongate member being laterally offset relative to the second elongate member, a first portion of the second elongate member being positioned within the first recess, a second portion of the second elongate member being laterally offset relative to the first elongate member; and (c) an end effector positioned at a distal end of the shaft assembly, the end effector including: (i) a first component coupled with the first elongate member, the first elongate member being operable to drive movement of the first component, and (ii) a second component coupled with the second elongate member, the second elongate member being operable to drive movement of the second component.

### Example 2

The apparatus of Example 1, the first component comprising a first jaw.

### Example 3

The apparatus of Example 2, the end effector further comprising a second jaw, the first jaw being movable relative to the second jaw, in response to movement of the first elongate member, to thereby transition the first jaw and the second jaw between an open state and a closed state the first jaw and the second jaw being operable to grasp tissue in the closed state.

### Example 4

The apparatus of Example 2, the second component comprising a knife, the knife being operable to sever tissue.

### Example 5

The apparatus of Example 4, the first jaw defining a slot, the knife being operable to translate along the slot in response to movement of the second elongate member.

### Example 6

The apparatus of any of Examples 1 through 5, the end effector further comprising one or more electrodes, the one or more electrodes being operable to apply radiofrequency energy to tissue.

### Example 7

The apparatus of any of Examples 1 through 6, the first elongate member defining a “U” shaped cross-sectional profile oriented in a first direction, the second elongate defining a “U” shaped cross-sectional profile oriented in a second direction.

### Example 8

The apparatus of Example 7, the second direction being opposite to the first direction.

### Example 9

The apparatus of any of Examples 7 through 8, the first elongate member and the second elongate member being positioned such that the “U” shaped cross-sectional profile of the first elongate member overlaps with “U” shaped cross-sectional profile of the second elongate member.

### Example 10

The apparatus of any of Examples 1 through 9, the shaft assembly defining a central longitudinal axis, the outer shaft being coaxial with the central longitudinal axis, the first elongate member being positioned within the outer shaft such that the first elongate member is radially centered about the central longitudinal axis.

### Example 11

The apparatus of Example 10, the second elongate member being positioned within the outer shaft such that the second elongate member is laterally offset relative to the central longitudinal axis.

### Example 12

The apparatus of any of Examples 1 through 11, the shaft assembly further comprising a third elongate member positioned within the outer shaft, the third elongate member being longitudinally fixed relative to the outer shaft.

### Example 13

The apparatus of Example 12, the third elongate member defining a third recess, the second portion of the first elongate member being positioned within the third recess of the third elongate member.

### Example 14

The apparatus of Example 13, the first portion of the second elongate member being positioned within the third recess of the third elongate member.

### Example 15

The apparatus of any of Examples 13 through 14, the third elongate member defining a fourth recess, the shaft assembly further comprising a wire disposed in the fourth recess, the wire providing a conduit for communication of radiofrequency energy to the end effector.

### Example 16

The apparatus of any of Examples 1 through 15, the body including a structure selected from the group consisting of a handle and a robotic system interface.

### Example 17

An apparatus comprising: (a) a body; (b) a shaft assembly extending distally relative to the body, the shaft assembly including: (i) an outer shaft defining a central longitudinal axis, (ii) a first elongate member movably positioned within the outer shaft, the first elongate member being operable to translate relative to the outer shaft, the first elongate member being positioned within the outer shaft such that the first elongate member is radially centered about the central longitudinal axis, (ii) a second elongate member movably positioned within the outer shaft, the second elongate member being operable to translate relative to the outer shaft and relative to the first elongate member, the second elongate member being positioned within the outer shaft such that the second elongate member is laterally offset relative to the central longitudinal axis at a first lateral side of the central longitudinal axis, and (iii) a third elongate member fixedly positioned within the elongate shaft, the third elongate member being positioned within the outer shaft such that the third elongate member is laterally offset relative to the central longitudinal axis at a second lateral side of the central longitudinal axis; and (c) an end effector positioned at a distal end of the shaft assembly, the end effector including: (i) a first jaw, (ii) a second jaw, the first elongate member being coupled with the first jaw to drive movement of the first jaw relative to the second jaw, and (iii) a knife, the second elongate member being coupled with the knife to drive movement of the knife relative to one or both of the first jaw or the second jaw.

### Example 18

The apparatus of Example 17, the first elongate member defining a first channel, the second elongate member defining a second channel, a first portion of the first elongate member being positioned within the second channel, a second portion of the first elongate member being laterally offset relative to the second elongate member, a first portion of the second elongate member being positioned within the first channel, a second portion of the second elongate member being laterally offset relative to the first elongate member.

### Example 19

A method comprising: (a) providing a knife assembly, the knife assembly including a body and a knife, the knife assembly being positioned relative to a longitudinal axis, the knife assembly including a body defining a recess; (b) moving a closure beam toward the knife assembly, along a first path that is transversely oriented relative to the longitudinal axis, such that a first portion of the closure beam is positioned in the recess defined by the body of the knife assembly, and such that a second portion of the closure beam is laterally offset relative to the body of the knife assembly; (c) moving an inner guide toward the closure beam, along a second path that is transversely oriented relative to the longitudinal axis, such that the closure beam is positioned in a recess defined by a body of the inner guide; (d) moving an outer tube over the knife assembly, the closure beam, and the inner guide, along the longitudinal axis, to thereby contain the knife assembly, the closure beam, and the inner guide within an interior of the outer tube, thereby forming a shaft assembly; and (e) coupling an end effector with the shaft assembly, the end effector including: (i) a first jaw, and (ii) a second jaw, the closure beam being coupled with the first jaw to drive movement of the first jaw relative to the second jaw, the knife of the knife assembly being operable to translate along one or both of the first jaw or the second jaw.

### Example 20

The method of Example 19, wherein the second path is substantially perpendicular to the first path.

### V. Miscellaneous

Any of the versions of the instruments described herein may include various other features in addition to or in lieu of those described above. While the examples herein are described mainly in the context of electrosurgical instruments, various teachings herein may be readily applied to a variety of other types of devices. By way of example only, the various teachings herein may be readily applied to other types of electrosurgical instruments, tissue graspers, tissue retrieval pouch deploying instruments, surgical staplers, surgical clip appliers, ultrasonic surgical instruments, etc. The teachings herein may be readily applied to any of the instruments described in any of the references cited herein. Any of the devices herein may also include one or more of the various features disclosed in any of the various references that are incorporated by reference herein. The teachings herein may thus be readily combined with the teachings of any of the references cited herein in numerous ways. Other types of instruments into which the teachings herein may be incorporated will be apparent to those skilled in the art in view of the teachings herein.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. The disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure.

The terms “proximal” and “distal” are defined herein relative to a surgeon, robotic arm, or other operator or structure grasping a surgical instrument having a distal surgical end effector. The term “proximal” refers the position of an element closer to the surgeon, robotic arm, or other operator or structure; and the term “distal” refers to the position of an element closer to the surgical end effector of the surgical instrument and further away from the surgeon or other operator or structure.

As used herein, an element or step recited in the singular and proceeded with the word “a” or “an” should be understood as not excluding plural of said elements or steps, unless such exclusion is explicitly stated. Furthermore, references to “one example” or “an example” are not intended to be interpreted as excluding the existence of additional examples that also incorporate the recited features. The use of “including,” “comprising,” or “having” and variations thereof herein is meant to encompass the items listed thereafter, and equivalents thereof, as well as additional items.

As used herein in the specification and claims, including as used in the examples and unless otherwise expressly specified, the terms “about” or “approximately” for any numerical values or ranges indicate a suitable dimensional tolerance, or other form of reasonable expected range, that allows the part or collection of components to function for its intended purpose as described herein. More specifically, “about” or “approximately” may refer to the range of values that are within ±10% of the recited value (e.g., “about 100” may refer to the range of values from 90 to 110, including 90, 110, 100, and all other values within the range of 90 and 110). Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein. The terms “approximately” and “about” are thus utilized herein to represent the degree by which a quantitative representation may vary from a stated reference without resulting in a change in the basic function of the subject matter at issue.

The term “substantially” is also utilized herein to represent the degree by which a quantitative representation may vary from a stated reference without resulting in a change in the basic function of the subject matter at issue. The term “substantially” shall therefore be understood to include a range of conditions or results that provide a functional equivalent to an explicitly stated condition or result. For instance, if a task is “substantially complete,” the result of the task having been substantially completed is functionally equivalent to the result that would have been achieved if the task had been perfectly completed. As another non-limiting example, a component that is “substantially straight” or “substantially flat,” an apparatus including a component that is “substantially straight” or “substantially flat” may provide a result or effect that is functionally equivalent to a result or effect that would be achieved by the same apparatus including the same component in a perfectly straight or perfectly flat configuration. The range implied by the term “substantially” should also be read to include the perfect result that is within that range. Thus, the term “substantially complete” shall be read as including “perfectly complete” while also including a range of completeness that is functionally equivalent to perfectly complete. As another example, terms such as “substantially straight” and “substantially flat” shall be read as including “perfectly straight” and “perfectly flat,” respectively; while also including a range of straightness or flatness that is functionally equivalent to perfectly straight or flat, respectively. As with the terms “approximately” and “about,” the term “substantially” may indicate a suitable dimensional tolerance, or other form of reasonable expected range, that allows a part or collection of components to function for its intended purpose as described herein.

The limitations of the following claims are not written in means-plus-function format and are not intended to be interpreted based on 35 U.S.C. §112(f), unless such claim limitations expressly use the phrase “means for” followed by a statement of function void of further structure.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

What is claimed is:

1. An apparatus comprising:
(a) a body;
(b) a shaft assembly extending distally relative to the body, the shaft assembly including:
(i) an outer shaft,
(ii) a first elongate member positioned within the outer shaft, the first elongate member being operable to translate relative to the outer shaft, the first elongate member defining a first recess, and
(ii) a second elongate member positioned within the outer shaft, the second elongate member being operable to translate relative to the outer shaft and relative to the first elongate member, the second elongate member defining a second recess,
a first portion of the first elongate member being positioned within the second recess, a second portion of the first elongate member being laterally offset relative to the second elongate member,
a first portion of the second elongate member being positioned within the first recess, a second portion of the second elongate member being laterally offset relative to the first elongate member; and
(c) an end effector positioned at a distal end of the shaft assembly, the end effector including:
(i) a first component coupled with the first elongate member, the first elongate member being operable to drive movement of the first component, and
(ii) a second component coupled with the second elongate member, the second elongate member being operable to drive movement of the second component.

2. The apparatus of claim 1, the first component comprising a first jaw.

3. The apparatus of claim 2, the end effector further comprising a second jaw, the first jaw being movable relative to the second jaw, in response to movement of the first elongate member, to thereby transition the first jaw and the second jaw between an open state and a closed state the first jaw and the second jaw being operable to grasp tissue in the closed state.

4. The apparatus of claim 2, the second component comprising a knife, the knife being operable to sever tissue.

5. The apparatus of claim 4, the first jaw defining a slot, the knife being operable to translate along the slot in response to movement of the second elongate member.

6. The apparatus of claim 1, the end effector further comprising one or more electrodes, the one or more electrodes being operable to apply radiofrequency energy to tissue.

7. The apparatus of claim 1, the first elongate member defining a “U” shaped cross-sectional profile oriented in a first direction, the second elongate defining a “U” shaped cross-sectional profile oriented in a second direction.

8. The apparatus of claim 7, the second direction being opposite to the first direction.

9. The apparatus of claim 7, the first elongate member and the second elongate member being positioned such that the “U” shaped cross-sectional profile of the first elongate member overlaps with “U” shaped cross-sectional profile of the second elongate member.

10. The apparatus of claim 1, the shaft assembly defining a central longitudinal axis, the outer shaft being coaxial with the central longitudinal axis, the first elongate member being positioned within the outer shaft such that the first elongate member is radially centered about the central longitudinal axis.

11. The apparatus of claim 10, the second elongate member being positioned within the outer shaft such that the second elongate member is laterally offset relative to the central longitudinal axis.

12. The apparatus of claim 1, the shaft assembly further comprising a third elongate member positioned within the outer shaft, the third elongate member being longitudinally fixed relative to the outer shaft.

13. The apparatus of claim 12, the third elongate member defining a third recess, the second portion of the first elongate member being positioned within the third recess of the third elongate member.

14. The apparatus of claim 13, the first portion of the second elongate member being positioned within the third recess of the third elongate member.

15. The apparatus of claim 13, the third elongate member defining a fourth recess, the shaft assembly further comprising a wire disposed in the fourth recess, the wire providing a conduit for communication of radiofrequency energy to the end effector.

16. The apparatus of claim 1, the body including a structure selected from the group consisting of a handle and a robotic system interface.

17. An apparatus comprising:
(a) a body;
(b) a shaft assembly extending distally relative to the body, the shaft assembly including:
(i) an outer shaft defining a central longitudinal axis,
(ii) a first elongate member movably positioned within the outer shaft, the first elongate member being operable to translate relative to the outer shaft, the first elongate member being positioned within the outer shaft such that the first elongate member is radially centered about the central longitudinal axis,
(ii) a second elongate member movably positioned within the outer shaft, the second elongate member being operable to translate relative to the outer shaft and relative to the first elongate member, the second elongate member being positioned within the outer shaft such that the second elongate member is laterally offset relative to the central longitudinal axis at a first lateral side of the central longitudinal axis, and
(iii) a third elongate member fixedly positioned within the elongate shaft, the third elongate member being positioned within the outer shaft such that the third elongate member is laterally offset relative to the central longitudinal axis at a second lateral side of the central longitudinal axis; and
(c) an end effector positioned at a distal end of the shaft assembly, the end effector including:
(i) a first jaw,
(ii) a second jaw, the first elongate member being coupled with the first jaw to drive movement of the first jaw relative to the second jaw, and
(iii) a knife, the second elongate member being coupled with the knife to drive movement of the knife relative to one or both of the first jaw or the second jaw.

18. The apparatus of claim 17, the first elongate member defining a first channel, the second elongate member defining a second channel, a first portion of the first elongate member being positioned within the second channel, a second portion of the first elongate member being laterally offset relative to the second elongate member, a first portion of the second elongate member being positioned within the first channel, a second portion of the second elongate member being laterally offset relative to the first elongate member.

19. A method comprising:
(a) providing a knife assembly, the knife assembly including a body and a knife, the knife assembly being positioned relative to a longitudinal axis, the knife assembly including a body defining a recess;
(b) moving a closure beam toward the knife assembly, along a first path that is transversely oriented relative to the longitudinal axis, such that a first portion of the closure beam is positioned in the recess defined by the body of the knife assembly, and such that a second portion of the closure beam is laterally offset relative to the body of the knife assembly;
(c) moving an inner guide toward the closure beam, along a second path that is transversely oriented relative to the longitudinal axis, such that the closure beam is positioned in a recess defined by a body of the inner guide;
(d) moving an outer tube over the knife assembly, the closure beam, and the inner guide, along the longitudinal axis, to thereby contain the knife assembly, the closure beam, and the inner guide within an interior of the outer tube, thereby forming a shaft assembly; and
(e) coupling an end effector with the shaft assembly, the end effector including:
(i) a first jaw, and
(ii) a second jaw, the closure beam being coupled with the first jaw to drive movement of the first jaw relative to the second jaw,
the knife of the knife assembly being operable to translate along one or both of the first jaw or the second jaw.

20. The method of claim 19, wherein the second path is substantially perpendicular to the first path.
